(19) United States
(12) Patent Application Publication
(10) Pub. No.: US 2012/0165676 A1
(43) Pub. Date: Jun. 28, 2012

(54) Neuravionic System for Life Support in High Performance Avionics

(76) Inventor: PHILIP CHIDI NJEMANZE, OWERRI (NG)

(21) Appl. No.: 12/975,383

(22) Filed: Dec. 22, 2010

(51) Int. Cl. A61B 8/06

(52) U.S. Cl. 600/454

(57) ABSTRACT

A system for life support in high performance avionics that utilizes cerebral blood flow velocity measurements and responses to real-time neuropsychological tests of brain function, to accomplish prevention of gravitational loss of consciousness, determination of cognitive state-of-being of the crewmember, regulation of autonomy-decision making level, while taking into account individualized +Gz-tolerance and cognitive abilities under +Gz-stress, comprising a transcranial Doppler device, attached to a microcomputer, operatively connected to the mainframe avionic computer.

### CROSS REFERENCE TO RELATED APPLICATION

Not applicable

### STATEMENT REGARDING FEDERALLY SPONSORED RESEARCH OR DEVELOPMENT

Not applicable

### REFERENCE TO MICROFICHE APPENDIX

Not Applicable

### REFERENCES CITED

### U.S. Pat. Nos.

| | | | |
|---|---|---|---|
| 3,780,723 | December 1973 | Van Paten et al. | 600/19 |
| 4,336,590 | June 1982 | Jacq et al. | 364/418 |
| 4,417,584 | November 1983 | Cathignol et al. | 128/663 |
| 4,736,731 | April 1988 | Van Patten | 600/20 |
| 4,817,633 | April 1989 | McStravick | 128/202.11X |
| 4,906,990 | March 1990 | Robinson | 340/945 |
| 5,121,744 | June 1992 | Njemanze | 128/202.11 |
| 6,390,979 | May 2002 | Njemanze | 600/438 |
| 6,547,737 | April 2003 | Njemanze | 600/454 |
| 6,663,571 | December 2003 | Njemanze | 600/504 |
| 6,773,400 | August 2004 | Njemanze | 600/454 |

### BACKGROUND OF THE INVENTION

The present invention provides a system for life support that utilizes cerebral blood flow velocity measurements and responses to real-time neuropsychological tests of brain function, to accomplish prevention of loss of consciousness and determination of cognitive state-of-being of the human subject. The invention finds application in high performance avionics where mental performance monitoring of the crewmember at the human-avionic computer interface, could be used for cognitive biometric identification of the crewmember, prevention of GLOC and regulation of autonomy-decision making level between the crewmember, autopilot and mission control center.

High performance avionic systems such as fighter aircrafts F-18, F-35 and spacecrafts, present the problem of countering the effects of +Gz acceleration. The U.S. Air Force introduced the Combined Advanced Technology Enhanced Design G Ensemble (COMBAT EDGE) Program for full scale development under the Tactical Life Support System Program. COMBAT EDGE is a G-protection system that provides the crewmember with increasing levels of breathing gas pressure as gravitational force increases, and has been described in a publication by Tripp L D, titled Combat Edge: a test subject perspective, published in SAFE Journal, 1991, volume 21, pages 21-25. This is achieved by using the G-valve outlet pressure to drive the breathing regulator, which in turn provides the crewmember with positive pressure breathing for G protection (PBG). However, the assessment of the benefits of PBG is constrained by lack of objective parametric measures of the effects of G forces on the crewmember's cerebral blood mean flow velocity (MFV). Currently, assessment of acceleration stress tolerance is based on time of endurance after reaching peak +Gz in a given G profile. However, such measurements do not necessarily reflect the state of cerebral perfusion and yet the adverse effects of high G on vision (gravitational loss of vision, G-LOV) and loss of consciousness (GLOC), both result from fall in cerebral perfusion as described in a publication by Glaister D H, titled The effects of long duration acceleration, in a book by, Ernsting J, King P, eds, titled Aviation medicine, London: Butterworths, 1988, pages 139-158. Indeed, Glaister maintains that, there is a direct relationship between applied acceleration and fall in cerebral perfusion.

Cerebral blood flow velocity could be measured using transcranial Doppler ultrasound sonography (TCD). The principles for measurement of blood flow velocity in cerebral vessels have been described in detail in a book edited by Aaslid R, entitled Transcranial Doppler Sonography, and published by Springer Verlag, Wien, 1989, on pages 39-50. The U.S. Pat. No. 4,417,584 to Cathignol et al. describes Doppler velocimeter for real-time display of blood flow velocities in a segment of a blood vessel. Applying non-invasive TCD ultrasound, we undertook studies to examine the variation of cerebral perfusion indexed by MFV during +Gz acceleration and PBG. In a study by Njemanze P C, Antol P J, Lundgren C E G, titled Perfusion of the visual cortex during pressure breathing at different high-G stress profiles published in Aviation Space and Environmental Medicine, 1993, volume 64, pages 396-400, the MFV in the right (RPCA) and left (LPCA) posterior cerebral arteries that supply blood to the visual cortex, were shown to drop in a centrifuge subject during exposure at various high G-profiles, but increased during exposure to PBG. The G profiles examined included gradual (GOR) of 0.1 G/s to 10 Gz for 10s and rapid onset rate (ROR) of 0.5 G/s to +6 Gz, sustained for a maximum of 3 minutes or to end-points given below; and ROR Simulated Aerial Combat Maneuver (SACM), with 1.04 G/s transitions, between +5 and +9 Gz, and with 10-s plateaus. The end-points for all profiles included successful profile completion, fatigue, failure to respond to buzzer signal, cardiac rhythm abnormalities, or visual light loss. During all the G profiles, the MFV declined with increasing +Gz with anti-G suit protection alone. The MFV increased in direct proportion with increase in +Gz acceleration with PBG. The mediating mechanisms for the effects of PBG may include improved gaseous exchange, direct effects on cerebral circulation, diminished sympathicoadrenal discharges and possible role of cardiopulmonary reflexes. Another study, by Njemanze P C, titled Critical limits of pressure flow relation in the human brain, published in Stroke, 1992, volume 23, pages 1743-1747, demonstrated that, the critical level of cerebral blood flow velocity for human consciousness is 50% of the MFV recorded at resting baseline in supine position.

The underlying mechanisms implicated in cerebral hypoperfusion during loss of consciousness have not been fully elucidated. However, major insights gained from studies using head-up tilt (HUT) induced syncope, differentiated two different types of mediating physiologic responses. In a publication by Njemanze P C, titled Cerebral circulation dysfunction and hemodynamic abnormalities in syncope during upright tilt test, published in the Canadian Journal of Cardiology, 1993, volume 9, pages 238-242, it was demonstrated that, there are two types of clinical unconsciousness, type I, neurogenic syncope, which implicates mechanisms of regulation of cerebral blood flow velocity in the human brain and was not accompanied by hypotension, and type II, cardiogenic syncope, which implicated mechanisms of cardiovascular regulation and was accompanied by hypotension. Furthermore, to clarify the role of cardiopulmonary reflexes, it was demonstrated in a publication by Njemanze P C, titled Isoproterenol induced cerebral hypoperfusion in a heart transplant recipient, published in Pacing and Clinical Electrophysiology (PACE), 1993, volume 16, 491-495, that in a heart transplant recipient with total deafferentation, type I neurogenic syncope occurred, thus cardiac reflexes must not play a role. Similarly, cardiopulmonary reflexes may not be implicated in type I neurogenic syncope as demonstrated in a heart-lung transplant recipient, as described in a publication by Njemanze P C titled Cerebrovascular dysautoregulation syndrome in heart-lung transplant recipient, published in Journal of Cardiovascular Technology, 1992, volume 10, pages 227-232. Conversely, cardiopulmonary reflexes could account for the systemic changes including fall in blood pressure, which accompany type II syncope, as described in publication by Njemanze P C titled Cerebral circulation dysfunction and hemodynamic abnormalities in syncope during upright tilt test, published in Canadian Journal of Cardiology, 1993, volume 9, pages 238-242. Furthermore, it was demonstrated that, the cerebral blood flow velocity waveforms in type I neurogenic syncope, was characterized by fall in both peak systolic velocity and end-diastolic velocity in a gradual stepwise fashion, while in type II cardiogenic syncope, the waveform showed only an end-diastolic velocity dropped in an abrupt manner. These changes in cerebral blood flow velocity may precede presentation of clinical symptoms, and systemic changes in heart rate and blood pressure, by as much as 2 minutes, as demonstrated by Njemanze P C, in a publication titled Cerebrovascular dysautoregulation syndrome complex—brain hypoperfusion precedes hypotension and cardiac asystole, published in Japanese Circulation Journal, 1994, volume 58, pages 293-297. It therefore follows that, monitoring of MFV using non-invasive TCD, could provide physiologic indices for human consciousness and could be applied for use in early detection of GLOC and activation of countermeasures including the pressurization of anti-G valve and controlling of air pressure for PBG.

However, the use of PBG presents the problem of both formation of gaseous and may be particulate microemboli, especially in the immediate post-pressurization period. Microembolic signals (MES) could be detected using TCD, which correspond to microbubbles or formed element particles composed of thrombotic or athromatous material, platelet-rich aggregates, or fat. The study by Russell D, Madden K P, Clark W M, Sanset P M, Zivin J A, titled Detection of arterial emboli using Doppler ultrasound in rabbits, published in Stroke, 1991, volume 22, pages 253-258, and the work by Markus H S, Brown M M, titled Differentiation between different pathological cerebral embolic materials using transcranial Doppler in an in vitro model, published in Stroke, 1993, volume 24, pages 1-5, detail the methodology for detection of MES using TCD. Similarly, TCD could be applied for monitoring and detection of MES during decompression for astronauts after extravehicular activity (EVA), and also in deep sea divers. It has been reported that, during clinical positive pressure breathing with 100% oxygen, there may be exponential decline in MES count, but after termination, there was subsequent increase in MES formation, attributed to the effect of oxygen on denitrogenation, as shown in a work by Georgiadis D, Wenzel A, Lehmann D, Lindner A, Zerkowski H R, Zierz S, Spencer M P, titled Influence of Oxygen Ventilation on Doppler Microemboli Signals in Patients With Artificial Heart Valves, published in Stroke. 1997, volume 28, pages 2189-2194.

Conventional methods do not apply physiologic monitoring, but rather use mechanical approaches. Prior art applied mechanically controlled anti-G suit valves or inertia valves, providing pressurization of air to the bladders at pressures proportional to acceleration. The U.S. Pat. No. 4,906,990 to Robinson describes a means to regulate the pressure within an anti-G suit, for use during space flight. The U.S. Pat. No. 3,780,723, to Van Paten et al. and U.S. Pat. No. 4,243,024 to Grosbie, et al., discloses electronic servo feedback mechanisms for quicker triggering of the anti-G valve. The latter inventions use the rate of change of acceleration, to set the threshold level for triggering the signal that initiates the inflation of the anti-G suit. Others like the disclosure in U.S. Pat. No. 4,336,590 to Jacq, et al., describes a microprocessor controlled anti-G valve that initiates inflation of the air bladders on control stick movement indicating imminent high acceleration. Early attempts have been made to apply monitoring of neurosensory system, for example, the U.S. Pat. No. 4,817,633, to McStravick, et al. discloses a light weight device to stimulate and monitor human vestibuloocular reflex. The U.S. Pat. No. 5,121,744 to Njemanze, discloses a physiologic anti-G suit modulator comprising a TCD device for sensing critical fall in cerebral perfusion and using the information to regulate the level of pressurization to the anti-G suit valve. The latter inventions introduced neurophysiologic monitoring of vestibuloocular reflex and cerebral perfusion. However, the invention of U.S. Pat. No. 5,121,744 to Njemanze, while it may ascertain physiologic recovery of cerebral blood flow velocity in the brain of the crewmember following a GLOC episode, falls short of providing insight into the cognitive state-of-being of the crewmember to respond adequately to tasks, and thus may not prevent the adverse effects of GLOC on cognition. One major drawback of conventional approaches is that, the use of physical or physiologic parameters rather than psychophysiologic parameters, thus does not take account individual differences in G-tolerance and cognitive abilities under +Gz stress.

The assessment of cognitive functions may include mental performance, facial processing, object processing, attention, vigilance, odor processing, color processing, motor processing, linguistic and nonlinguistic processing, accomplished by noninvasive measurement of MFV in major cerebral arteries using techniques of functional transcranial Doppler (fTCD) and functional transcranial Doppler spectroscopy. The U.S. Pat. No. 6,390,979 to Njemanze, describes a device using fTCD for assessment of mental performance. However, the U.S. Pat. No. 6,390,979 to Njemanze falls short of integrating GLOC monitoring with real-time neuropsychological test of brain function of the crewmember in-flight, which is crucial for life support of the crewmember. The monitoring of brain function in real-time in high-performance avionics is critical to mission goals, since without certification of a good mental state-of-being, the crewmember may not accomplish the mission objectives. The feasibility for real-time monitoring of mental performance using non-invasive fTCD has been demonstrated by Njemanze P C, in an article titled Cerebral lateralisation and general intelligence: Gender differences in a transcranial Doppler study, published in Brain and Language, volume 92, pages 234-239. The latter work, utilized Raven Progressive Matrices (RPM), a test of general intelligence, to establish that, successful outcome was associated with lateralization of MFV to the right middle cerebral artery (RMCA) but not to the left middle cerebral artery (LMCA), in men. While in women, successful task outcome was associated with LMCA lateralization but not RMCA. While in both genders, unsuccessful outcome was associated with bilateral activation or no lateralization. In other words, mental performance monitoring could be realized in real-time using fTCD. Similarly, real-time fTCD monitoring could be accomplished during linguistic and non-linguistic processing. During audio linguistic and classical music (with lyrics) stimulations, it has been shown that, there are specific patterns of lateralization depending on handedness and vascular physiologic dominance as described in detail in a publication by Njemanze P C, Cerebral lateralization in linguistic and non-linguistic perception: analysis of cognitive styles in the auditory modality, published in Brain and Language, 1991, volume 41, pages 367-380. Furthermore, in the visual modality, language stimulation has been shown to elicit cerebral blood flow velocity changes, as described in detail in a publication by Njemanze P C, titled Cerebral lateralization in random letter task in the visual modality: a transcranial Doppler study, published in Brain and Language, 1996, volume 53, pages 315-325. Altogether, real-time TCD monitoring of responses to mental performance, linguistic and non-linguistic stimulations are feasible using non-invasive fTCD in a high performance avionic environment.

Prior arts have attempted to examine application of fTCD for real-time monitoring of singular neuropsychological functions. The U.S. Pat. No. 6,773,400 to Njemanze describes a device that uses fTCD for assessment of facial and object recognition. The detailed description of the assessment of facial and object processing using fTCD and fTCDS has been given in a publication by Njemanze P C, titled Cerebral lateralisation for facial processing: Gender-related cognitive styles determined using Fourier analysis of mean cerebral blood flow velocity in the middle cerebral arteries, published in Laterality, 2007, volume 12, pages 31-49. In simulated microgravity, there are changes in lateralization of brain blood flow velocity during facial processing in men and women, as described in detailed in a publication by Njemanze P C, titled Asymmetry in cerebral blood flow velocity with processing of facial images during head-down rest, published in Aviation Space and Environmental Medicine, 2004, volume 75, pages 800-805.

Furthermore, the use of fTCD for real-time monitoring of odor processing has been demonstrated in prior art. The U.S. Pat. No. 6,663,571 to Njemanze describes a device that uses fTCD for assessment of odor processing in human subjects with capability for identification of responses associated with a target odor. Similarly, the human brain response to color stimulation has been demonstrated using fTCD technique as described by Njemanze P C, Gomez C R, and Horenstein S, in an article titled Cerebral lateralisation and color perception: A transcranial Doppler study, published in Cortex, 1992, volume 28, pages 69-75, as well as work by Njemanze P C, in an article titled Asymmetry of cerebral blood flow velocity response to colour processing and hemodynamic changes during −6 degrees 24-hour head-down bed rest in men, published in Journal of Gravitational Physiology, 2005, volume 12, pages 33-41. Furthermore, a detailed description of the application of fTCDS for determination of color and luminance processing in the human brain has been given by Njemanze P C, in an article titled Asymmetric neuroplasticity of color processing during head down rest: a functional transcranial Doppler spectroscopy study, published in Journal of Gravitional Physiology, 2008, volume 15, pages 49-59. It follows that, application of fTCD for real-time monitoring of the effects on MFV due to a target odor, color and luminance processing is feasible.

Similarly, real-time monitoring of the effects of motor function on cerebral perfusion is feasible using fTCD. The effects of fine finger movements on MFV changes in the RMCA and LMCA has been assessed using fTCD, in a study by Njemanze P C, in an article titled Cerebral lateralization for motor tasks in simulated microgravity. A transcranial Doppler technique for astronauts, published in Journal of Gravitational Physiology, 2002, volume 9, pages 33-34. The effects of straining maneuver on MFV during PBG, has been assessed in a publication by Njemanze P C, Antol P J, Lundgren C E G, titled Perfusion of the visual cortex during pressure breathing at different high-G stress profiles published in Aviation Space and Environmental Medicine, 1993, volume 64, pages 396-400. It is feasible to integrate real-time monitoring of the effects of motor function on MFV using fTCD.

The study of selective attention of the crewmember is crucial to mission objectives using high performance aircrafts. The fTCD technique has been used to measure cerebral mean flow velocity during the Trail Making Tests (TMT), a means of selective attention and complex cognitive functioning as demonstrated in a publication by Misteli M, Duschek S, Richter A, Grimm S, Rezk M, Kraehenmann R, Boeker H, Seifritz E, Schuepbach D, titled Gender characteristics of cerebral hemodynamics during complex cognitive functioning, published in Brain and Cognition, 2011, (in press). Studies with other modalities such as functional magnetic resonance (fMRI) demonstrate anatomical correlates in the brain during TMT, as described in a publication by Zakzanis, K., Mraz R, Graham S J, titled An fMR1 study of the Trail Making Test, published in Neuropsychologia, 2005, volume 43, pages 1878-1886. The TMT has two parts of the paradigm, namely Part A and B. In the Trail Making Test, Part A (TMT-A), 25 numbers are depicted that have to be connected in an incrementing way (1, 2, 3 . . . 25) as fast as possible. The test is used to assess graphomotor speed, visual scanning and selective attention, referred to here as ‘selective attention’. In the Trail Making Test, Part B (TMT-B), numbers (1 to 13) and letters (A to L) must be linked in a mutually and incrementing fashion, and it provides information on mental flexibility and executive functioning, and here referred to as ‘complex cognitive functioning’, as described in detail in a publication by Tombaugh T N, titled Trail Making Test A and B: Normative data stratified by age and education, published in Archives of Clinical Neuropsychology, 2004, volume 19, pages 203-214. The tasks are displayed on the visor and the crewmember could use mouse pointer or other means to solve the tasks as quickly and accurately as possible.

The digit vigilance task is a commonly used test of attention and psychomotor speed, alertness, and mental processing capacity, using a rapid visual tracking task applied in real-time. The reliability, validity and sensitivity have been examined in a publication by Kelland D Z, Lewis R F, titled The digit vigilance test: reliability, validity, and sensitivity to diazepam, published in Archives of Clinical Neuropsychology, 1996, volume 11, pages 339-344. Others have shown right lateralization pattern of MFV using fTCD during vigilance as described in a publication by Helton W S, Hollander T D, Warm J S, Tripp L D, Parsons K, Matthews G, Dember W N, Parasuraman R. Hancock P A, titled The abbreviated vigilance task and cerebral hemodynamics, published in Journal of Clinical and Experimental Neuropsychology, 2007, volume 29, pages 545-552.

The present invention would require application of a portable TCD, with an automated probe insonation device integrated into the headgear or helmet of the crewmember. Prior art have demonstrated that both portable TCD and an automatic intelligent TCD probe headgear are feasible. The application of a portable TCD to monitoring of MFV for mental performance has been described in detail in U.S. Pat. No. 6,390,979 to Njemanze. Similarly, the U.S. Pat. No. 6,663,571 to Njemanze describes a portable TCD for odor processing, and the U.S. Pat. No. 6,773,400 to Njemanze describes a portable TCD for facial and object processing. The U.S. Pat. No. 6,547,737 to Njemanze, describes an intelligent TCD probe for automated insonation of cerebral arteries.

At present, identification of persons allowed into high security computer networks as well as into high-performance aircraft systems is accomplished by static biometric systems, usually including finger printing, facial photographs and iris identification. However, these systems are static and lack the capacity to monitor dynamic changes in a crewmember. The trend of terrorism developing in recent years is such that, persons officially certified to logon to high security networks including high performance aircraft systems may turn to commit terrorist acts by themselves. At present, there are no dynamic monitoring methods that, will detect changes in the mental state-of-being and logout the crewmember. The present invention accomplishes dynamic monitoring of cognitive state-of-being in a crew member, by monitoring MFV responses to specific neuropsychological tests. The resulting subject specific trend is designated as the mental performance signature. In other words, the mental performance signature is the trend of MFV in response to real-time neuropsychological tests, which has high reproducibility, specificity and sensitivity. A tolerance limit for variation in MFV could be developed on repeated testing, and hence forms a performance envelope specific for a particular crewmember. The pattern associated with best performance is determined, and the borderline regions between good/best on one hand, and bad performance on the other, define the borders of the mental performance envelope. The latter could be used as a means of identification of the crewmember on the network, and hence designated as cognitive biometric identification. Cognitive biometric identification utilizes mental performance signature of a crewmember to activate human-avionic computer interface system. The system is configured to shut out the crewmember during periods when his performance falls outside the acceptable performance envelope. The rationale is that, anxiety and attention deficits would interfere with rational judgment in a crewmember, intending to engage in premeditated terrorist activity, and hence alter his usual mental performance signature.

The occurrence of brain death of the crewmember in high performance aircraft mishaps is difficult to confirm. Therefore search and rescue operations may unnecessarily be undertaken at a significant cost in human lives, instead of search and recovery operations. In clinical settings, brain death could be detected using TCD. The TCD characteristics of brain death has been described by Ringelstein E B, in an article titled Transcranial Doppler monitoring in a book titled Transcranial Doppler Sonography edited by Aaslid R, published by Springer-Verlag Wien, 1986, pages 147-162. The phenomenon is an oscillating movement of the blood column within the extracranial and/or the major intracranial arteries. The waveform flow profiles are dependent on cardiac output, and may be very sharp and pulsatile, or quite contrary, damped with sluggish acceleration and deceleration of the blood column. A reflux phenomenon during the later systole following antegrade injection of the blood into the vascular tree is diagnostic in every case. The present invention provides for brain death detection during mishaps, which could be helpful for deciding on search and recovery operations, rather than presumed search and rescue.

It is desirable to have a system that performs a psychophysiologic assessment of the cognitive state-of-being of the crewmember in response to prevailing aircraft or spacecraft microenvironment, for determination of the state of mental and physical fitness for mission specific tasks, cognitive biometric identification, and regulation of autonomy-decision making level between the crewmembers, autopilot and mission control center.

An object of the present invention is to provide a means of using the cerebral blood flow velocity responses to +Gz acceleration to regulate the pressurization of anti-G suit bladders, based on individualized +Gz-tolerance.

An object of the present invention is to provide a means of using cerebral blood flow velocity responses to +Gz acceleration for regulation of the pressure for positive pressure breathing at G, based on individualized +Gz-tolerance.

An object of the present invention is to provide a means of using cerebral blood flow velocity responses to neuropsychological tests to determine the crewmember mental state-of-being, and communicating, the said crewmember state-of-being to a remote computer at the mission control center for manual or automated regulation of autonomy decision-making level between crewmembers, autopilot and mission control center.

Another object of the present invention is to determine the facial processing mechanism for assessment of the cognitive state-of-being in a crewmember.

A further object of the present invention is to determine target object recognition in a crewmember for assessment of the cognitive state-of-being in a crewmember.

A further object of the present invention is to determine selective attention in a crewmember for assessment of the cognitive state-of-being in a crewmember.

A further object of the present invention is to determine vigilance in a crewmember for assessment of the cognitive state-of-being in a crewmember.

An object of the present invention is to determine the odor processing mechanism for assessment of the cognitive state-of-being in a crewmember.

An object of the present invention is to determine responses to motor stimulation for assessment of the cognitive state-of-being in a crewmember.

An object of the present invention is to determine responses to linguistic and non-linguistic stimuli for assessment of the cognitive state-of-being in a crewmember.

An object of the present invention is the determination of a specific mental performance signature for a crewmember, used as a means for cognitive biometric identification on the computer network of the avionic system.

A further object of the present invention is the detection of MES during PBG and decompression in a crewmember.

A further object of the present invention is the detection of brain death signal in a crewmember after a mishap.

These and other objects may become apparent to those skilled in the art upon reviewing the description of the invention as set forth hereinafter, in view of its drawings.

### SUMMARY OF THE INVENTION

The present invention provides a system for life support that utilizes cerebral blood flow velocity measurements and responses to real-time neuropsychological tests of brain function, to accomplish prevention of loss of consciousness and determination of mental state-of-being of the human subject. The invention finds application in high performance avionics where mental performance monitoring of the crewmember at the human-avionic computer interface, could be used for cognitive biometric identification of the crewmember, prevention of GLOC and regulation of autonomy-decision making level between the crewmember, autopilot and mission control center.

The special embodiment of this invention is illustrated in the specification, includes block and schematic diagrams for the format of the instrumentation, and how the system functions is shown, by way of example. The subject refers to the human crewmember, by way of example. The system comprises a TCD device that is connected to microcomputer hardware with appropriate software, and operatively and wirelessly connected to the avionic main frame computer. The present invention uses a portable TCD device with pulsed wave 1-2 MHz transducer or probe. The TCD probe is placed on the acoustic window of the temporal bone above the zygomatic are on both sides of the head. The probes could be initially manually set to insonate the cerebral arterials from both sides, for example the RMCA and LMCA, or right (RACA) and left (LACA) anterior cerebral arteries, or (RPCA) and LPCA or right (RICA) and left (LICA) internal carotid arteries, respectively. An automated probe headgear integrated into the helmet could be affixed. The coordinates for the cerebral vessels are stored in memory of the microcomputer of the device for each individual subject. The device automatically insonates the cerebral arteries, by recalling the initial coordinates stored in memory or by applying coordinates derived from brain imaging (computer tomography and magnetic resonance imaging) maps of cerebral vessels for corresponding bitemporal head size, using bilateral transcranial Doppler probes placed on the temporal bone on both sides of the head. The headgear has a reservoir for ultrasonic gel and tubing that automatically delivers the ultrasonic gel to the surface of the probes. The ultrasound signals are obtained from the main stem of the major cerebral arteries, for example, from the RMCA and LMCA at a depth of 50 mm from the surface of the probe. The baseline MFV data are obtained at rest and during neuropsychological test battery, that may include motor processing, selective attention, vigilance, facial processing, target object recognition, mental performance (intelligence) processing, color/luminance processing, odor processing, linguistic and non-linguistic (classical music) processing. The visual neuropsychological tests are displayed on the visor of the helmet during administration. The system derives a preflight best mental performance envelop which could be compared to prior data for cognitive biometric identification and later for in-flight comparison. On commencement of flight, in-flight data including responses to neuropsychological tests are acquired, which are compared to pre-flight data. The crewmember may execute several in-flight maneuvers such as ROR, GOR, and SACM, during which TCD monitoring is continued. If the MFV approaches set low threshold values or drop below it suddenly, then the microcomputer sends a signal to the avionic mainframe computer to perform a number of actions that may include activation of pressurization of the anti-G suit bladders, increase in pressure of the PBG and downgrading the autonomy-decision-making level of the crewmember. The autonomy to make decisions on mission-related critical issues may include but not limited to choice of flight path, selection of targets, and authority to continue or call off the mission. During increased PBG, the microcomputer activates the MES detection software, so that if, MES counts are increased, then a signal is sent to the mainframe computer to regulate the pressure during PBG and in the decompression phase. When the MFV returns to normal, the crewmember undergoes real-time neuropsychological test battery once again, and the results are compared to preflight data. If the MFV values are within normal range, and good performance envelope is restored, then the system upgrades the autonomy-decision-making level of the crewmember.

### BRIEF DESCRIPTION OF SEVERAL VIEWS OF THE DRAWINGS

FIG. 1 shows a crewmember in a helmet with head mounted display and PBG fittings affixed with the headgear of the present invention.

FIG. 2 shows an astronaut in a space suit on a manned maneuvering unit for extravehicular activity affixed with the present invention.

FIG. 3 shows the details of the transducer housing built into the walls of the headgear or helmet of the crewmember.

FIG. 4 shows the schematic diagram of the present invention.

FIG. 5 in panels A-D shows an example of the display on the visor of the crewmember, and panel E shows the crewmember affixed with the present invention while in a cockpit of a high performance aircraft.

FIG. 6 shows the multi-functional integrated system of the present invention.

FIG. 7A shows the first part of the functional flow chart of the present invention.

FIG. 7B shows the second part of the functional flow chart of the present invention for certification of full psychophysiologic recovery.

### DETAILED DESCRIPTION OF THE INVENTION

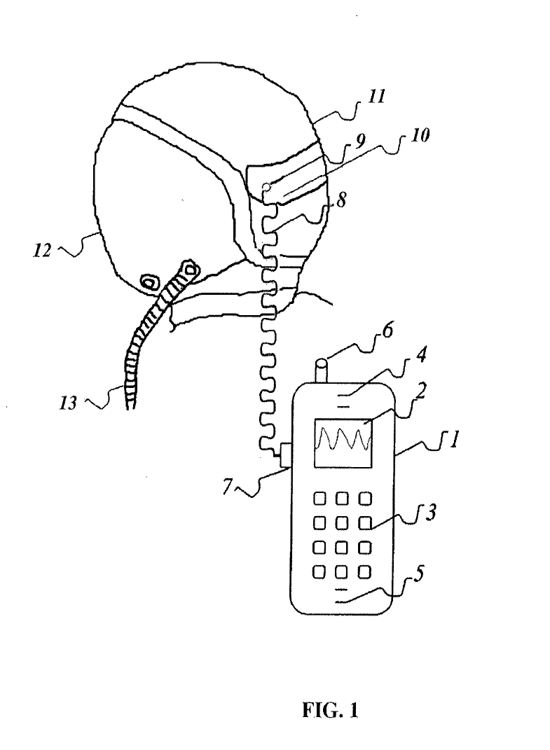

FIG. 1 shows a crewmember in a helmet with head mounted display and PBG fittings affixed with the headgear of the present invention. As shown, the transcranial Doppler device 1 connected to a microcomputer with spectrum analyzer 2, input keyboard 3, loudspeaker 4, microphone 5, and an aerial 6 for wireless communication. The port 7 is provided for attachment of the transducer cable 8 from the transducer 9, placed within a probe housing 10, integrated into the helmet 11. Similar miniaturized TCD device could be obtained from a company called DWL (Sipplingen, Germany), and similar automated headgear for positioning the transducers could be obtained as model ROBOTOC2MD from a company called Multigon (Yonkers, N.Y.), by way of example. The flight parameters, TCD parameters and neuropsychological test battery are shown on the head mounted display visor 12. The PBG fittings and tube 13 are attached in front.

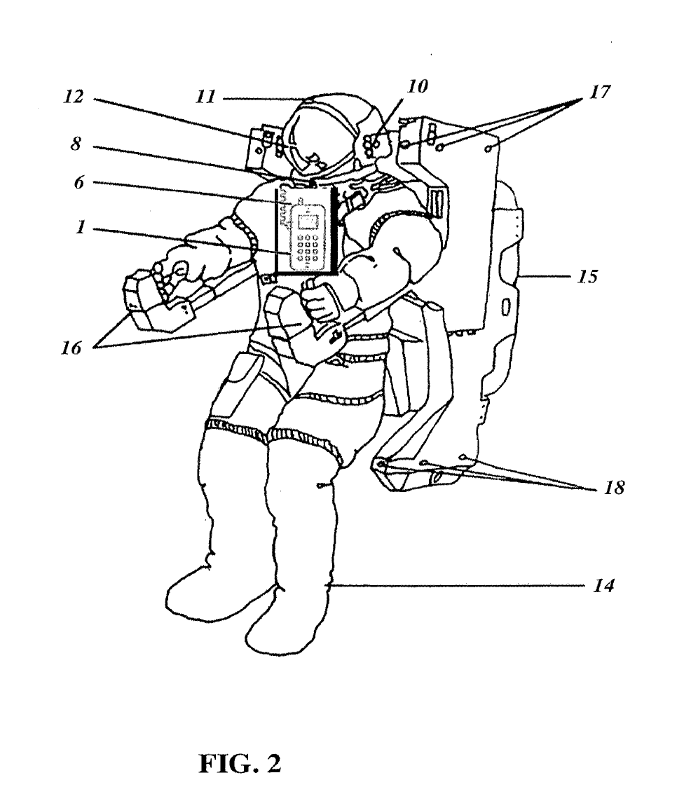

FIG. 2 shows an astronaut in a space suit on a manned maneuvering unit for extravehicular activity affixed with the present invention. The space suit 14, comprising a helmet 11 with visor 12. The transducer housings 10 are placed on both sides of the temples incorporated within the helmet 11. The transducer cable 8 connects to the TCD device 1, which has an aerial 6 for wireless communication with the space craft mainframe computer and the microcomputer that controls the space suit pressure, the extravehicular mobility unit 15, the acceleration controls 16, of the manned maneuvering unit with thruster nozzles 17 and 18.

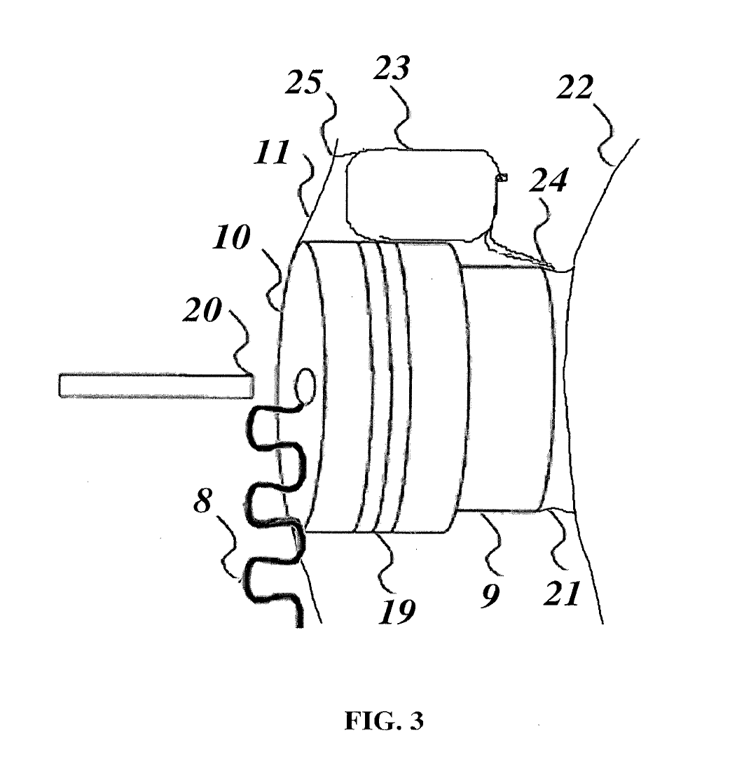

FIG. 3 shows the details of the transducer housing built-in within the walls of the headgear or helmet of the crewmember. The transducer cable 8 connects the transducer 9 to the TCD device. The transducer 9 has a backing material 19, with a detachable handle 20, attached only for first-time positioning of the probe for insonation of the major cerebral arteries, for example the RMCA ad LMCA at a depth of 50 mm. The transducer 9 surface is coupled to the skin using a gel pad 21 placed on skin of the temporal bone 22 on both sides, and gel supplied from a reservoir tank 23 built into the helmet wall. The gel could be expressed automatically from the tank 23 through the drainage tubing 24 during increased acceleration, and could be refilled through an injector site 25 on the exterior of the helmet.

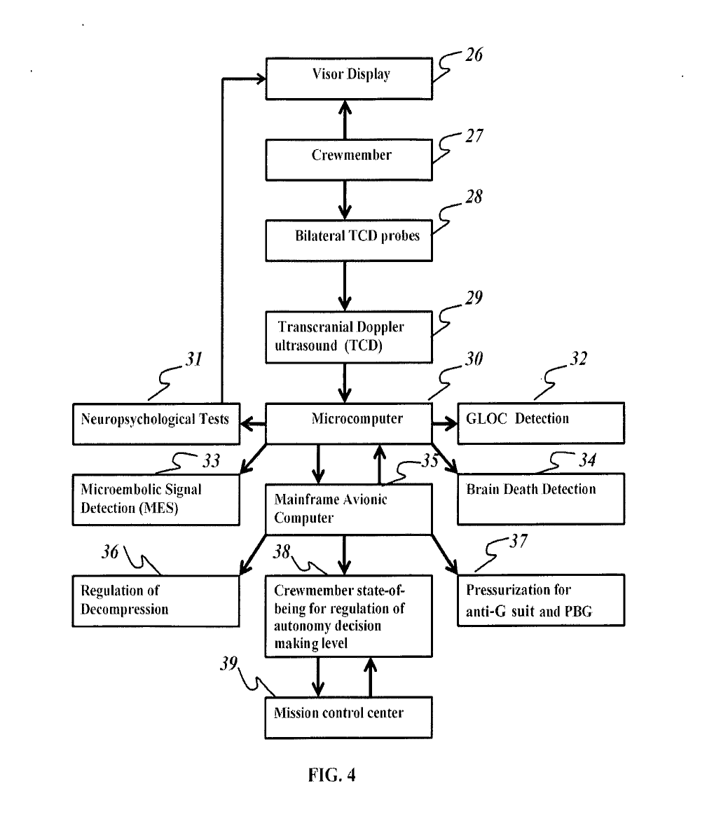

FIG. 4 shows the schematic diagram of the present invention. The crewmember 27 is fitted with the head mounted display visor 26, with attached headgear holding bilateral TCD probes 28 of the transcranial Doppler ultrasound device (TCD) 29, which is connected to the microcomputer 30 with appropriate software for sending control signals. Such a microcomputer is available from Intel®, Xeon® processor 3400 series, by way of example. The microcomputer 30 hosts the software for real-time administration of neuropsychological test battery 31, which are displayed on the visor 26. Such neuropsychological test battery 31 including the Raven Progressive Matrices (RPM) for general intelligence testing could be obtained from the American Psychological Association, Washington D.C. The microcomputer software uses the MFV values and waveform patterns for GLOC detection 32, and also performs microemboli detection 33. Such a TCD software could be obtained from DWL (Sipplingen, Germany), by way of example. The microcomputer also checks the MFV and waveforms for patterns associated with brain death 34. The microcomputer 30 operatively connects to the avionic mainframe computer 35 by direct connection or by wireless, to exchange information on the crewmember-state-of-being. The avionic mainframe computer such as those with F/A-22 Common Integrated Processor could be obtained from Raytheon Company, Waltham, Mass. The avionic mainframe computer will then regulate decompression 36, pressurization for the anti-G suit and PBG 37, as well as assesses overall crewmember state-of-being, and communicating, the said crewmember state-of-being 38 to a remote computer at the mission control center 39 for manual or automated regulation of autonomy decision-making level between crewmembers, autopilot and commanders at mission control center.

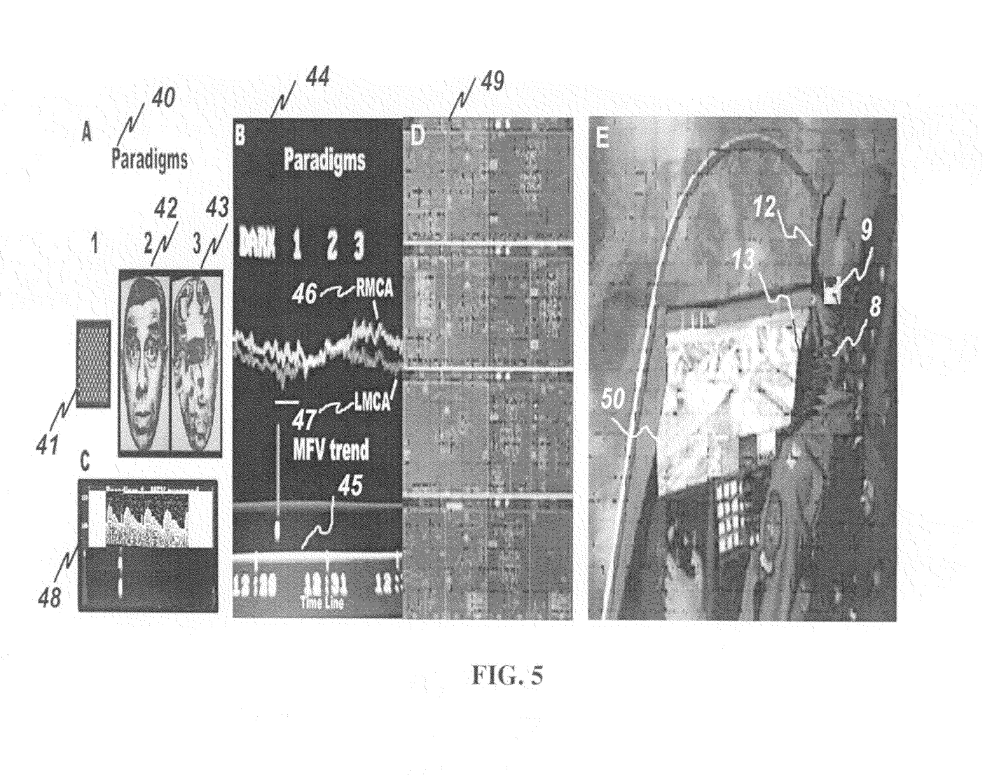

FIG. 5 in panels A-D shows an example of the display on the visor of the pilot, and panel E shows the pilot affixed with the present invention in a cockpit of a high performance aircraft. The neuropsychological tests could be displayed when necessary on the helmet-head mounted display visor 12, as shown 40, with Paradigm 1 as an object 41, Paradigm 2 as a whole face 42, and Paradigm 3 as a disarranged face 43. Panel B 44, shows the time-line 45 used for tracking time of stimulation and response, in MFV trend in the RMCA 46, LMCA 47, which form the stimulus response MFV profile used for deriving mental performance signature and envelope, by way of example. The flow velocity waveforms are shown in panel C 48. The flight parameters 49 are also displayed simultaneously in panel D. Panel E, shows the outlook of the TCD transducer 9, transducer cable 8, PBG fittings 13 and the helmet-head mounted display visor 12, as seen within the cockpit 50.

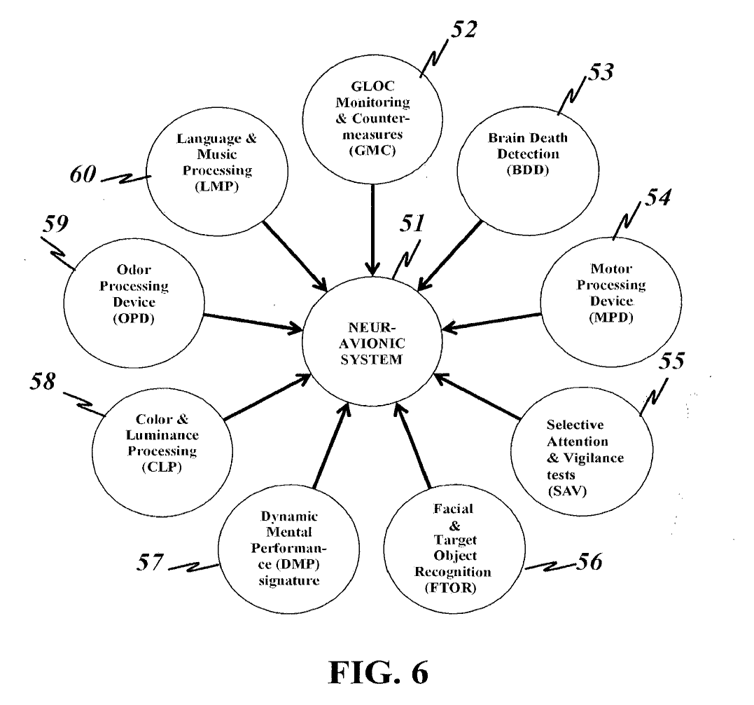

FIG. 6 shows the multi-functional integrated system of the present invention. The Neuravionic system 51 comprises TCD device integrated with neuropsychological testing software and materials for multi-functional monitoring of GLOC and countermeasures (GMC) 52, brain death detection (BDD) 53, motor processing device (MPD) 54, selective attention and vigilance tests (SAV) 55, facial and target object recognition (FTOR) 56, dynamic mental performance signature (DMP) 57, color and luminance processing (CLP) 58, odor processing device (OPD) 59, language and music processing (LMP) 60.

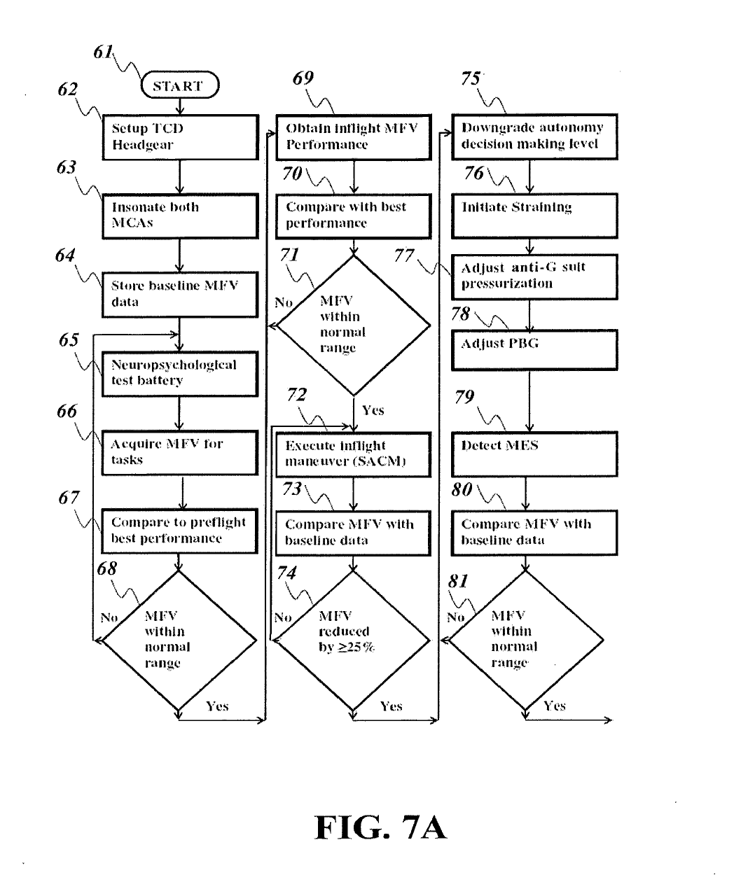

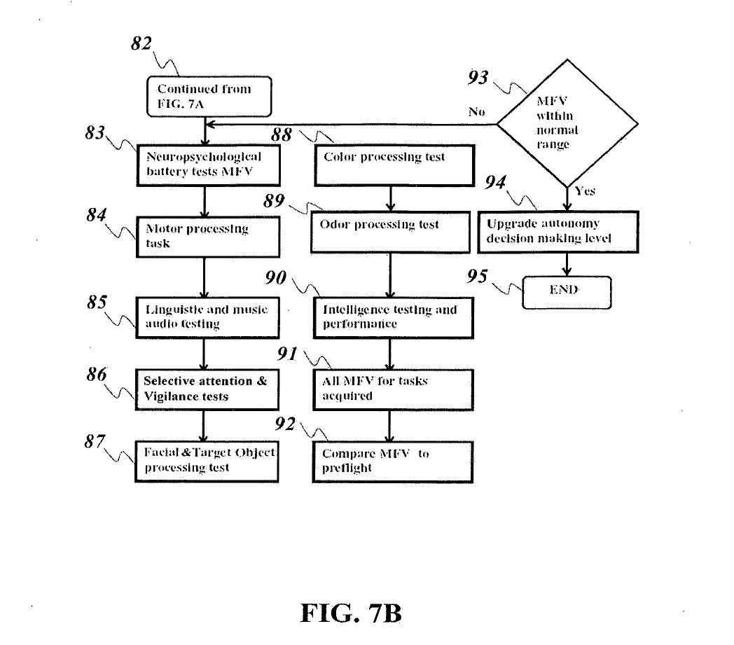

FIG. 7A shows the first part of the functional flow chart of the present invention. At the start of flight preparation 61, the crewmember is fitted with the TCD device with transducers attached to the temporal bone on both sides of the head 62, for insonation of the RMCA and LMCA 63, by way of example. The baseline MFV data are recorded and stored 64. The neuropsychological test battery 65 are administered in real-time, with simultaneous recording of the MFV in the MCAs in response to tasks 66. The MFV data are compared to that archived for best performance 67 for the crewmember. If the MFV values are not within the normal range 68, then a rerun of the neuropsychological tests 65 may be necessary, to ascertain the psychophysiologic state of the crewmember to undertake the mission. However, if MFV values are within normal range 68, then the program proceeds to the next step. The initial in-flight MFV performance data 69 including responses to select real-time neuropsychological tests are acquired, and compared to best performance data in memory 70. If the MFV values are not within normal range 71, a real-time performance test rerun may be required. Otherwise, the program proceeds 71 to the next step. The crewmember may execute flight maneuvers similar to SACM 72, while being monitored in real-time with the TCD device, and the measured MFV values are compared with baseline 73. If the MFV is not reduced by 25% or more 74, the crewmember may continue flight maneuvers. However, if the MFV values are reduced by 25% or more, then the avionic mainframe computer downgrades the autonomy decision-making level of the crewmember 75. The crewmember initiates straining maneuvers 76, while the avionic mainframe computer adjusts the anti-G suit pressure 77, and increases PBG pressurization 78. The microcomputer software analyzes the flow velocity envelop for detection of MES 79, while continuing comparison of MFV values with that of baseline 80, for assessment of the effectiveness of the G-protection countermeasures. If the MFV values remain below normal range 81, then the cycle is repeated from step 75. Otherwise, with MFV values restored to normal range 81, the system proceeds to certify full psychophysiologic recovery of the crewmember through detailed steps illustrated in FIG. 7B.

FIG. 7B shows the second part of the functional flow chart of the present invention for certification of full psychophysiologic recovery of the crewmember, continued from FIG. 7A 82. The MFV is recorded during administration of neuropsychological test battery 83, which would be illustrated by way of example only. The tests could begin with a simple motor processing task 84 such as finger movements. Then followed by a linguistic task such as music with lyrics, and then non-linguistic stimulation with classical music without lyrics 85. Tests of selective attention such as TMT-A and TMT-B and tests of vigilance 86. A facial processing task involving focused attention to a whole face, and then a mental arrangement of a disarranged face could also be used, and an target object processing and recognition task 87, which may include perception and then recognition of a target object, could be administered. This could also be followed by a color processing task 88, involving passive perception of colors red, blue and yellow, by way of example. The odor processing could be tested by using familiar odorants 89, or odor from known gases used in combat, or burning electrical wiring. The performance/intelligence task 90 may involve use of Raven Progressive Matrices or its variants, by way of example. The choice of real-time neuropsychological test battery may depend on time constraints and purpose. A simplified quick test may serve the purpose, and must have proven reproducibility, sensitivity and specificity. A combination neuropsychological test battery that combines major neurocognitive domains, drawn from a combination of motor processing, linguistic/non-linguistic processing, facial processing, object processing, color processing, odor processing, and general intelligence/performance processing, may be preferred. When all MFV values elicited by tasks are acquired 91, they are compared to preflight baseline 92. If the MFV values are not within the normal range 93, then there could be a rerun of the tests. Otherwise, with normal MFV values 93, the avionic mainframe computer upgrades the autonomy-decision-making level of the crewmember 94. The latter will bring the program to an end of the cycle 95.

Having thus described the invention, what is claimed and desired to be secured by Letters Patent is:

1. A system for life support that utilizes cerebral blood flow velocity measurements and responses to real-time neuropsychological tests of brain function, to accomplish prevention of loss of consciousness and determination of mental state-of-being of a human subject, comprising:
a transcranial Doppler device, attached to a microcomputer, operatively connected to a mainframe computer.

2. The invention of claim 1 and further including means for display of the transcranial Doppler parameters, waveforms, and neuropsychological tests.

3. The invention of claim 1 and further including headgear, means for automated insonation of the cerebral arteries, by recalling the initial coordinates stored in memory or by applying coordinates derived from brain imaging maps of cerebral vessels for corresponding bitemporal head size, using bilateral transcranial Doppler probes placed on the temporal bone on both sides of the head, said headgear, has an ultrasonic gel reservoir with tubing and gel pad, means for automatic delivery of ultrasonic gel to the surface of the probes.

4. The invention of claim 1 and further including means for using cerebral blood flow velocity response to neuropsychological tests for determination of a specific mental performance signature of a subject, for use in cognitive biometric identification on the computer network, as well as determination of mental state-of-being for regulation of autonomy decision-making level between subjects and mission control center.

5. A system for life support in high performance avionics that utilizes cerebral blood flow velocity measurements and responses to real-time neuropsychological tests of brain function, to accomplish prevention of gravitational loss of consciousness and determination of mental state-of-being of the crewmember, comprising:
a transcranial Doppler device, attached to a microcomputer, operatively connected to the avionic mainframe computer.

6. The invention of claim 5 and further including means for detection of microembolic signals for regulation of decompression pressure.

7. The invention of claim 5 and further including means for detection of cerebral blood flow velocity signals indicating brain death.

8. The invention of claim 5 and further including means for detection of decrease in cerebral blood flow velocity indicating impending loss of consciousness.

9. The invention of claim 5 and further including means of using the cerebral blood flow velocity responses to +Gz acceleration to regulate the pressurization of anti-G suit bladders, based on individualized +Gz-tolerance.

10. The invention of claim 5 and further including means of using cerebral blood flow velocity responses to +Gz acceleration for regulation of the pressure for positive pressure breathing at G, based on individualized +Gz-tolerance.

11. The invention of claim 5 and further including means of using cerebral blood flow velocity responses to neuropsychological tests to determine the crewmember mental state-of-being, and communicating, the said crewmember state-of-being to a remote computer at the mission control center for manual or automated regulation of autonomy decision-making level between crewmembers, autopilot and mission control center.

12. A system for life support in high performance avionics that utilizes cerebral blood flow velocity measurements and responses to real-time neuropsychological tests of brain function, to accomplish prevention of gravitational loss of consciousness, determination of cognitive state-of-being of the crewmember, regulation of autonomy-decision making level, while taking into account individualized +Gz-tolerance and cognitive abilities under +Gz-stress, comprising:
a transcranial Doppler device, attached to a microcomputer, operatively connected to the mainframe avionic computer.

13. The invention of claim 12 wherein the neuropsychological battery includes motor processing tasks.

14. The invention of claim 12 wherein the neuropsychological battery includes facial and target object recognition tasks.

15. The invention of claim 12 wherein the neuropsychological battery includes color and luminance processing tasks.

16. The invention of claim 12 wherein the neuropsychological battery includes tests for selective attention and vigilance.

17. The invention of claim 12 wherein the neuropsychological battery includes tests for language and music processing.

18. The invention of claim 12 wherein the neuropsychological battery includes tests for odor processing.

19. The invention of claim 12 wherein the neuropsychological battery includes tests for intelligence processing.

20. The invention of claim 12 wherein the system is electrically powered by means of a rechargeable battery source or mains.
